(19) United States
(12) Patent Application Publication
(10) Pub. No.: US 2026/0232610 A1
(43) Pub. Date: Aug. 13, 2026

(54) COMPOSITION COMPRISING GUANIDINOACETIC ACID FOR USE IN THE TREATMENT AND/OR PROPHYLAXIS OF A COCCIDIOSIS INDUCED CONDITION IN POULTRY

(71) Applicant: Evonik Operations GmbH, Essen (DE)

(72) Inventors: Rose WHELAN, Ambrosden (GB); Martina KLÜNEMANN, Frankfurt (DE); Juliano Cesar DE PAULA DORIGAM, Hanau (DE)

(73) Assignee: Evonik Operations GmbH, Essen (DE)

(21) Appl. No.: 19/471,236

(22) PCT Filed: Mar. 25, 2024
(86) PCT No.: PCT/EP2024/057905

§ 371 Date: Sep. 30, 2025

(30) Foreign Application Priority Data
Apr. 3, 2023 (EP) 23166269.3

(51) Int. Cl. A61K 31/195; A23K 10/18; A23K 20/142; A23K 50/75; A61P 31/04

(52) U.S. Cl. CPC A61K 31/195; A23K 10/18; A23K 20/142; A23K 50/75; A61P 31/04

(57) ABSTRACT

A composition suitable for the treatment and/or prophylaxis of a coccidiosis induced condition in poultry. The composition comprises guanidinoacetic acid, a salt of guanidinoacetic acid, or a mixture thereof. The coccidiosis may be caused by Eimeria spp. or Clostridium perfringens, and the coccidiosis induced condition may be bacterial enteritis, necrotic enteritis, diarrhea, or lesion.

Necrotic enteritis (NE) is one of the most relevant enteric diseases in poultry and leads to high costs in the poultry industry worldwide (see Skinner et al., Avian Diseases 2010, 54(4): 1237-40). It is the case of USD 6 billion annual losses in global poultry production and this controllable disease is on the rise. One reason is the voluntary or legally required reduction of antibiotics in animal production. This trend is driven by the increasing occurrence of antimicrobial resistance, as well as by consumer demand. Another reason is the reduction of ionophores which, besides their activity against coccidia, also show efficacy against clostridia. When anticoccidial live vaccines are used, the application of these ionophores is not possible and clostridia/necrotic enteritis increase (Williams, 2005).

While this is a widespread problem in all poultry, for broilers in particular necrotic enteritis and coccidiosis are a significant health problem.

Necrotic enteritis is a multifactorial disease. While its etiology is under research, the leading theory is as follows (see Fathima et al., Microorganisms 2022, 10(10): 1958; Abd El-Hack et al., Poultry Science 2022, 101(2): 101590): Factors like Coccidiosis or heat stress weaken the gut integrity. Coccidiosis is an endemic disease in the commercial broiler industry and is caused by development and reproduction of parasitic Eimeria species, which can infect a specific area of the gut and cause tissue damage in the intestinal epithelial cells. The disruption of the intestinal epithelial cells leads to the impaired absorption of nutrients and increased intestinal permeability (see Teng et al., Poultry Science 2020, 99(9): 4203-4216), which may lead to a high risk of bacterial infections. Additionally, the active immune suppression utilized by the parasite to remain in the host, makes the host more susceptible to secondary infections, for example with avian-specific Clostridium perfringens leading to necrotic enteritis.

Since coccidiosis is often part of the disease development, coccidiostats are used to control Eimeria infection (see Mesa-Pineda et al., Frontiers in Veterinary Science 2021, 8:87653) and antibiotics to control pathogenic bacteria, thus avoiding development of enteric diseases. Like antibiotics, chemical anticoccidials or coccidiostats induce development of resistance in Eimeria species. Therefore, it is recommended to stop their use at least once per year for an entire broiler growth cycle. Instead, live vaccines are used containing attenuated strains of Eimeria species. However, vaccination often incurs the risk of a mild coccidiosis. Since the ban of antimicrobial growth promoters and the rise of “no antibiotics ever” movement, the impact of necrotic enteritis increases further. To this end, many recent studies of necrotic enteritis have focused on finding different ways to control the disease, and to understand its pathogenesis. Premixes or technical products that can improve gut health or directly have an impact on the infection but are not antibiotics, may play an important role in controlling the disease and the negative commercial effects.

In general, necrotic enteritis occurs in broiler chickens of 2 to 6 weeks of age. In subclinical forms, it is characterized by impaired digestion. Clinical forms lead to severe problems and increased flock mortality in a very short time.

The clinical form of the necrotic enteritis is characterized by acute, dark diarrhea resulting in wet litter and suddenly increasing flock mortality of up to 1% per day after appearance of the first clinical signs (Ducatelle and Van Immerseel, 2010), sometimes summing up to mortality rates of 50% (Van der Sluis, 2013). The birds have ruffled feathers, lethargy, and inappetence.

Necropsy typically shows ballooned small intestines with a roughened mucosal surface, lesions, and brownish (diphtheritic) pseudo-membranes. There is a lot of watery brown, blood-tinged fluid, and a foul odor during post-mortem examination. The liver is dark, swollen, and form, and the gall bladder is distended (Hofacre et al., 2018).

In the case of per-acute necrotic enteritis, birds may die without showing any preliminary signs.

When birds suffer from the subclinical form, chronic damage to the intestinal mucosa and an increased quantity of mucus in the small intestine lead to impaired digestion and absorption of nutrients resulting in poor growth performance.

The deteriorated feed conversion and the resulting decreased performance become particularly noticeable around day 35 of age. As feed contributes approximately 65 to 70% of the input cost to produce a broiler chicken, poor feed conversion increases production costs and significantly influences profitability. Often, due to a lack of clear symptoms, this subclinical disease remains untreated and permanently impacts the efficiency of production.

Accordingly, there was still a need for a way for treating any type of coccidiosis induced condition in poultry. It was found that this problem is solved by a composition comprises guanidinoacetic acid, a salt of guanidinoacetic acid, or a mixture of any of these.

One object of the present invention is therefore a composition for use in the treatment and/or prophylaxis of a coccidiosis induced condition in poultry, wherein the composition comprises guanidinoacetic acid, a salt of guanidinoacetic acid, or a mixture of any of these.

Said composition is administered to poultry having a coccidiosis induced condition.

The term poultry is used in the context of the present invention to denote any kind of domesticated bird, captive raised for its utility. Examples for poultry are domestic fowls, including chickens (or broilers and layers), turkeys, geese, quails, and ducks, raised to produce meat or eggs. Preferably, the term poultry refers to chickens or broilers in the context of the present invention.

Supplementation with guanidinoacetic acid leads to a higher creatine availability in all tissue cells not only muscle tissue and therefore to a higher cellular energy availability in all cells. Furthermore, phosphorylated creatine acts as an energy buffer in cellular stress conditions and shifts the energy production away from glycolysis to the more efficient oxidative phosphorylation pathway. In addition, it leads to a higher expression of creatine kinase. Therefore, supplementation with guanidinoacetic acid improves energy digestibility by minimizing energy loss, energy availability for the immune response in immune cells and leads to a more favorable metabolic profile in the response to Eimeria infection or necrotic enteritis.

Coccidiosis is caused by development and reproduction of parasitic Eimeria species, which can infect a specific area of the gut and cause tissue damage in the intestinal epithelial cells. In addition, the active immune suppression utilized by the parasite to remain in the host, makes the host more susceptible to secondary infections, for example with avian-specific Clostridium perfringens leading to necrotic enteritis. However, the administration of guanidinoacetic acid supports poultry growth through improvement in energy efficiency in poultry that may be impaired due to the parasite infection or secondary induced inflammation. Especially, administration of guanidinoacetic acid improves the limited mitochondrial energy which is highly efficient for the bird's well-being. Hence, administration of the guanidinoacetic acid in particular improves broiler growth and resistance to a multifactorial necrotic enteritic challenge involving Eimeria spp. and Clostridium perfringens pathogens.

In one embodiment of the composition for use according to the present invention the coccidiosis is caused by Eimeria spp. and/or Clostridium perfringens.

In another embodiment of the composition for use according to the present invention the coccidiosis induced condition is one or more of bacterial enteritis, necrotic enteritis, diarrhea, and lesion.

Guanidinoacetic acid is soluble in water but compared to some amino acids such as glycine or arginine it has a rather low solubility in water. Nevertheless, the solubility of guanidinoacetic acid in water can be increased by transferring this compound into an acid addition salt. Suitable acid addition salts of guanidinoacetic acid can be formed with hydrogen chloride, sulfuric acid, and phosphoric acid.

In a further embodiment of the composition for use according to the present invention the salt of guanidinoacetic acid is an acid addition salt.

In a preferred embodiment of the composition for use according to the present invention the salt of guanidinoacetic acid is guanidinoacetic acid hydrogen chloride, guanidinoacetic acid hydrogen sulfate, guanidinoacetic acid hydrogen phosphate, or a mixture of any of these.

In principle, the composition according to the present invention is not subject to any limitations regarding the content of guanidinoacetic acid. Rather, the content of guanidinoacetic acid in the composition according to the present invention is more or less given by the national or regional registration regulations for feed stuff.

In an embodiment the composition for use according to the present invention comprises up to 1,500 ppm of guanidinoacetic acid, a salt of guanidinoacetic acid, or a mixture of any of these. Preferably, the composition according to the present invention comprises up to 1,200 ppm of guanidinoacetic acid, a salt of guanidinoacetic acid, or a mixture of any of these.

In addition to guanidinoacetic acid, the composition according to the present invention may comprise further components, in particular components which are beneficial for poultry health. Efficient animal production is a balancing act of nutrition, health, and animal welfare, especially when reducing or eliminating antibiotic utilization. In this context ensured intestinal health is absolutely essential. Challenges such as diseases, environmental factors, and feed ingredient quality, can negatively impact the intestinal microbial balance, leading to slower growth and reduced performance, which in turn result in economic losses. Microbiota imbalance (dysbiosis) but also overgrowth of Clostridium perfringens enables growth of opportunistic bacteria such as Escherichia coli, which can result in different disorders. These disorders, such as diarrhea, wet litter, necrotic enteritis, leaky gut (impaired gut barrier function), intestinal inflammation (colitis) and poor immune status finally impair the animal's feed efficiency, growth, and their wellbeing. It is therefore beneficial that the composition according to the present invention further comprises a probiotic.

In another embodiment the composition for use according to the present invention further comprises a probiotic.

It was found that a probiotic further improves the benefits of the composition according to the present invention. In particular, it was found that guanidinoacetic acid and an additional probiotic synergistically improve the growths of coccidiosis infected poultry and the resistance to a multifactorial necrotic enteritis challenge involving Eimeria spp. and Clostridium perfringens pathogens.

In a preferred embodiment of the composition for use according to the present invention the probiotic comprises spores of Bacillus spp.

A probiotic comprising spores of Bacillus subtilis, such as Evonik's GutCare®, is considered to directly inhibit the bacterial component, i.e., Clostridium perfringens, while stabilizing the microbiome and reducing leaky gut induced bacterial translocation.

A probiotic comprising spores of Bacillus amyloliquefaciens, such as Evonik's EcoBiol®, improves health and production conditions of animals and help producers to improve quality of their products profitability of their business and solve sustainability challenges. In detail, feed supplemented with a probiotic comprising spores of Bacillus amyloliquefaciens, such as Evonik's EcoBiol®, supports microbial balance in the gut. Said probiotic helps to reduce production cost through improved feed conversion and time reduction to slaughter.

The composition according to the present invention can further comprise one of the aforementioned two types of probiotics alone or in combination.

In a further preferred embodiment of the composition for use according to the present invention the probiotic comprises spores of Bacillus amyloliquefaciens and/or Bacillus subtilis.

For example, the probiotic comprises a strain selected from B. subtilis (e.g., DSM 32315 or DSM 32540), B. amyloliquefaciens (e.g., CECT 5940) and/or a mixture of any of these.

In principle, the composition according to the present invention can be administered to poultry in any phase, a multitude of phases, or all phases of feeding poultry, i.e., in any phase, a multitude of phases, or all phases of their lifetime. The lifetime of laying hens can be divided into a pre-layer phase and three production phases or three production periods, i.e., the initial production phase, the grower juvenile production phase, and the nesting phase, sometimes also called pre-layer phase and phases I to III. The lifetime of captive-raised birds for meat production can be divided into the three phases of starter, grower, and finisher phase. For example, the whole lifetime of chicken can amount to 39 days, of which the days (d) from d-0 to d-10 are called the starter phase, the days from d-10 to d-21 are called the grower phase and the days from d-21 to d-39 are called the finisher phase. Birds raised for egg production have respectively also different growth and feeding periods. In principle, the composition according to the present invention being administered to poultry having a coccidiosis induced condition, e.g., coccidiosis infected poultry, is not limited to any specific phase or period in the lifetime of poultry. Therefore, the composition can be administered to poultry having a coccidiosis induced condition, e.g., coccidiosis infected poultry, at any conceivable time point in or during any of the phases, i.e., in or during the starter, grower and/or finisher phase. Notwithstanding, it is preferred to administer the composition to poultry having a coccidiosis induced condition, e.g., coccidiosis infected poultry, in or during the finisher phase.

In one embodiment the composition for use according to the present invention is administered to poultry in any phase, a multitude of phases, or all phases of their lifetime.

It was observed that the daily weight gain of coccidiosis infected birds receiving the composition according to the present invention was significantly higher than that of coccidiosis infected birds receiving an antibiotic via the drinking water in the challenge period from day 16 to day 22 and overall, from day 0 to day 42.

In another embodiment the composition for use according to the present invention is administered to poultry starting from the beginning of the starter phase until slaughter or starting from the beginning of the grower phase until slaughter or any other phase of life.

In the grower phase, coccidiosis infected birds receiving the composition according to the present invention gained approximately 9 g/day more than the negative control and overall, they gained approximately 2 g/day more.

In a further embodiment the composition for use according to the present invention is administered to poultry in the grower phase.

In principle, the composition according to the present invention is not subject to a specific state of aggregation or matrix.

In an embodiment the composition for use according to the present invention is a liquid or a solid composition.

When the composition according to the present invention is a solid composition, it is preferred that it is an effervescent tablet. In that case the composition comprises a carbon dioxide producing compound and a gas releasing compound. Preferably, the equivalent ratio of the gas releasing compound to the carbon dioxide producing compound is 1:1 or more, and the molar ratio of the carbon dioxide producing compound to the guanidinoacetic acid is 1:1 or more.

This composition is particularly suitable for water supply application, preferably drinking water application because the carbon dioxide producing compound reacts with the gas releasing compound in the presence of water to release carbon dioxide. As a result, the dissolution process of the guanidinoacetic acid is greatly accelerated.

In order to provide or produce carbon dioxide, it is preferred that the carbon dioxide producing compound is a salt of carbonic acid.

In an embodiment of the composition according to the present invention the carbon dioxide producing compound is therefore an alkali carbonate, an alkaline earth carbonate, an ammonium carbonate, an alkali hydrogen carbonate, an alkaline earth hydrogen carbonate, an ammonium hydrogen carbonate, or a mixture of any of these.

In a preferred embodiment of the composition according to the present invention the carbon dioxide producing compound is a sodium hydrogen carbonate, a potassium hydrogen carbonate, an ammonium hydrogen carbonate, or a mixture of any of these.

In the case of an effervescent tablet, the gas releasing compound reacts with the carbon dioxide producing compound in the presence of water to give carbon dioxide. In principle, the underlying reaction is therefore an acid-base reaction, the hydroxonium ions (H₃O⁺) provided by the gas releasing compound react with the carbonate (CO₃²⁻) and/or hydrogen carbonate ions (HCO₃⁻) under formation of carbonic acid (H₂CO₃). However, the thus released carbonic acid is not thermodynamically stabile and easily disintegrates to carbon dioxide and water, which leads to the sparkling effect and the thus accelerated dissolution of the guanidinoacetic acid.

In order to allow for the best possible release of carbon dioxide, it is preferred that the gas releasing compound is a stronger (or even much stronger) acid in terms of pKₐ value(s) than the carbon dioxide producing compound, in particular the salt of carbonic acid. This condition is typically met when the gas releasing compound in the composition is an acid. By comparison, the carbonate in the salt of carbonic acid is a base. Carbonic acid has the pka values pkₐ₁=6.35 and pKₐ₂=10.33. Therefore, any gas releasing compound suitable for use in the composition according to the present invention should have pKa values lower than those of carbonic acid.

In an embodiment of the composition according to the present invention the gas releasing compound is an acid, e.g., an inorganic acid, an organic acid, or a mixture of any of these.

Preferably, the acid has one or more pKa values lower than 6.35. This requirement is met by a variety of organic acids, such as citric acid (pKₐ₁=3.13, pKₐ₂=4.76 and pKₐ₃=6.4), tartaric acid (pKₐ₁=2.98, and pKₐ₂=4.34) and malic acid (pKₐ₁=3.46, and pKₐ₂=5.10).

The use of a solid organic acid allows to provide the composition according to the present invention in solid form. A solid form of the composition according to the present invention is the most concentrated form of the said composition and therefore requires much less space than the liquid form, i.e., a solution of the said composition. It is therefore preferred that the composition according to the present invention is a solid composition.

In a preferred embodiment of the composition according to the present invention the gas releasing compound is therefore a solid organic acid.

Preferably, the gas releasing compound is citric acid, tartaric acid, malic acid, or a mixture of any of these.

In the context of the present invention the term equivalent ratio therefore denotes the ratio of the gas releasing compound to the carbon dioxide producing compound, which is required to give one (or more) molecule(s) of carbon dioxide. For example, where the gas releasing compound is citric acid with 3 carboxylic acid groups and the carbon dioxide producing compound is sodium hydrogen carbonate (NaHCO₃), 3 equivalents of citric acid react with 3 equivalents of sodium hydrogen carbonate to give 3 equivalents of carbon dioxide. Here, the equivalent ratio of the gas releasing compound to the carbon dioxide producing compound is 1:1, or in short 1. In another example, where the gas releasing compound is citric acid with 3 carboxylic acid groups and the carbon dioxide producing compound is sodium carbonate (Na₂CO₃), 6 equivalents of citric acid react with 3 equivalents of sodium carbonate to give 3 equivalents of carbon dioxide. Here, the equivalent ratio of the gas releasing compound to the carbon dioxide producing compound is 2:1, or in short 2.

Preferably, the equivalent ratio of the gas releasing compound to the carbon dioxide producing compound ranges from 1:1 to 3:1 in the composition according to the present invention.

In order to guarantee that carbon dioxide is produced or provided as completely as possible, it is preferred that the equivalent ratio of the gas releasing to the gas producing compound is at least 1.05:1.

When the diet according to the present invention is a solid matrix, it is preferred that said diet is a diet for poultry.

Another object of the present invention is a diet for use in the treatment and/or prophylaxis of a coccidiosis induced condition in poultry, wherein the diet comprises the composition according to the present invention.

### EXAMPLES

The in vivo study aimed to test the beneficial effects of GAA against necrotic enteritis (NE). General procedures and details of a floor pen study to determine the efficacy of GAA in the prevention and aid against experimentally induced NE in broilers are described below.

The experimental design consisted of 3 treatments: the first consisted of a basal diet as negative control (IUC), the second was a positive control with the same basal diet but birds received an antibiotic via the drinking water (20 mg/kg BW/day administered for 5 days) (ITC), and the last was the same basal diet with supplemental guanidino acetic acid (GAA) at 0.12% (1.2 g/kg) but no antibiotics in the drinking water. Feed treatments were applied in all rearing phases (starter phase from day 0 till day 16, grower phase from d 16 till day 23, and finisher phase from d 23 till day 42). Table 1 below summarizes the formulation and nutrient composition of the basal diets.

TABLE 1
Formulation and nutrient composition of the basal
diets in the starter, grower and finisher phase.

| | Starter | Grower | Finisher |
|---|---|---|---|
| Item (%, as-fed) | (d 0-d 16) | (d 16-d 23) | (d 23-d 42) |
| wheat | 39.29 | 44.19 | 46.15 |
| soybean meal, 48% | 33.62 | — | 24.54 |
| corn | 20.00 | — | 20.00 |
| soybean oil | 3.38 | 10.00 | 5.98 |
| animal fat (lard) | — | 6.61 | — |
| rye | — | 7.50 | — |
| wheat bran | — | 5.00 | — |
| full fat soybeans | — | 5.00 | — |
| fishmeal, 70% | — | 30.0 | — |
| limestone | 1.37 | — | 1.19 |
| (CaCO₃) | | | |
| monocalcium phosphate | 0.66 | — | 0.35 |
| Ca(H₂PO₄)₂ | | | |
| salt (NaCl) | 0.20 | — | 0.22 |
| sodium bicarbonate NaHCO₃ | 0.26 | 0.19 | 0.50 |
| vitamin and mineral premix | 0.50 | 0.50 | 0.50 |
| choline chloride, 60% | 0.02 | — | 0.01 |
| phytase (500 FTU) | 0.01 | 0.005 | 0.01 |
| DL-Met (99%) | 0.29 | — | 0.24 |
| L-Lys * HCl | 0.25 | — | 0.22 |
| L-Thr (98.5%) | 0.09 | — | 0.08 |
| L-Val | 0.06 | — | 0.02 |

| | Starter | Grower | Finisher |
|---|---|---|---|
| Formulated nutrients | (d 0-d 16) | (d 16-d 23) | (d 23-d 42) |
| crude protein | 22.72 | 29.38 | 19.87 |
| ME, kcal/kg | 3000 | 3257 | 3200 |
| SID Met + Cys | 0.88 | 0.97 | 0.76 |
| SID Lys | 1.21 | 1.71 | 1.00 |
| SID Thr | 0.77 | 0.95 | 0.65 |
| SID Arg | 1.34 | 1.53 | 1.10 |
| SID Val | 0.96 | 1.20 | 0.80 |

The NE challenge was implemented in the grower phase and all birds in this phase were challenged. Feed in start and finisher phase was a corn-soy-wheat diet typical in northern Europe, which is easily digestible and does not contain many anti-nutritional factors. The grower diet included a large proportion of fishmeal, wheat bran, and rye, resulting in a diet with high crude protein content. Those conditions and raw ingredients are known to be aversive to gut health. Additionally, in days 14 and 16, the broilers received a 10-fold overdose of Paracox-8 live Eimeria vaccine by oral administration. On each of days 18 to 21, the broilers also received Clostridium perfringens inoculations.

Each treatment was replicated in 12 pens with 18 male Ross 308 broilers each in a completely randomized block design. Feed formulation of diets was based on Ross 308 broilers requirements for energy, amino acids, and macro-minerals. Diets were provided as crumble for the starter phase and as pellets for the grower and finisher phase. The chickens were fed ad libitum and had free access to water during the whole experiment. Birds were vaccinated in the hatchery against Newcastle disease. No other commercial vaccines were given during the study.

On days 21 and 22, four chicken per pen were euthanized for intestinal lesion scoring (adopted from Timbermont et al., Avian Pathology 2010, 39:117-221). The same chickens were also scored for typical lesions for coccidiosis according to the method of Johnson and Reid (Johnson and Reid, Experimental Parasitology 1970, 28(1): 30-36) with a score from 0 (no lesions) to 4 (severe lesions) for the species relevant to broilers described in this scoring system. Chickens in this study were scored for E. acervuline and E. maxima).

For statistical evaluations a Dunnett test was used, comparing antibiotics and GAA treatments against untreated challenged control (UTC). The tables below summarize the resulting means and p-values for GAA treatment. Bold letters highlight significant differences to IUC, also for antibiotics treatment.

TABLE 1
Summary of the growth performance parameters for
all treatments and the corresponding p-values.

| Growth | | | | |
|---|---|---|---|---|
| performance | | | | |
| parameters | IUC | ITC | GAA | p-value GAA |
| BW_D0 | 47 | 47 | 46 | 0.441 |
| BW_D16 | 725 | 714 | 711 | 0.141 |
| BW_D22 | 1139 | 1312 | 1161 | 0.5962 |
| BW_D34 | 2467 | 2745 | 2463 | 0.992 |
| BW_D42 | 3376 | 3714 | 3332 | 0.753 |
| DWG_D0-D16 | 42.2 | 41.4 | 41.0 | 0.072 |
| DWG_D16-D22 | 63.4 | 100.6 | 72.2 | 0.057 |
| DWG_D22-D34 | 72.1 | 82.2 | 75.3 | 0.499 |
| DWG_D34-D42 | 110.7 | 114.5 | 108.7 | 0.929 |
| DWG_D0-D42 | 60.3 | 70.0 | 62.6 | 0.187 |
| DFI_D0-D16 | 47.3 | 46.6 | 45.9 | 0.045 |
| DFI_D16-D22 | 81.5 | 100.2 | 84.4 | 0.444 |
| DFI_D22-D34 | 115.8 | 125.0 | 117.3 | 0.847 |
| DFI_D34-D42 | 199.2 | 209.5 | 191.0 | 0.370 |
| DFI_D0-D42 | 113.7 | 115.9 | 112.1 | 0.233 |
| FCR_D0-D16 | 1.12 | 1.13 | 1.12 | 0.951 |
| FCR_D16-D22 | 1.31 | 1.00 | 1.18 | 0.017 |
| FCR_D22-D34 | 1.63 | 1.52 | 1.56 | 0.418 |
| FCR_D34-D42 | 1.82 | 1.88 | 1.77 | 0.885 |
| FCR_D0-42 | 1.90 | 1.66 | 1.80 | 0.067 |
| NE related mortality | 13.33 | 0 | 3.33 | 0.044 |

Table 1 summarizes the growth performance parameters of the broilers for all treatments and all rearing phases, including the p-values. In general, infected antibiotics treated birds (ITC) performed better than infected untreated birds (UTC). Large differences in body weight (BW), daily weight gain (DWG), and feed conversion ratio (FCR) show that antibiotics are an efficient effective treatment of NE in broilers. The daily weight gain (DWG) of GAA supplemented birds was significantly higher than that of IUC birds in the challenge period from day 16 to d22. In the grower phase, GAA supplemented birds gained approximately 9 g/day more than the negative control and overall, they gained approximately 2 g/day more. There was no significant effect on the daily feed intake (DFI) (p>0.05) in any period for GAA treated birds over untreated birds except a small effect in the pre-challenge period. As DWG was higher and DFI was similar, FCR of GAA treated birds was significantly lower than that of untreated birds in the challenge period and overall, as well. Finally, overall, it was seen that the percentage of NE related mortalities was strongly reduced with GAA supplementation and on par with antibiotics treated control.

TABLE 2
Summary of the gut health scores for all treatments
and the corresponding p-values.

| Gut health scores | day | IUC | ITC | GAA | p-value GAA |
|---|---|---|---|---|---|
| NE score | 21 | 3.42 | 0.40 | 3.52 | 0.906 |
| E. acervulina | 21 | 0.15 | 0.02 | 0.08 | 0.536 |
| E. maxima | 21 | 0.81 | 0.38 | 0.52 | 0.038 |
| TMLS | 21 | 0.96 | 0.40 | 0.60 | 0.0268 |
| NE percentage | 21 | 88 | 13 | 85 | 0.957 |
| NE score | 22 | 3.25 | 0.52 | 2.33 | 0.001 |
| E. acervuline | 22 | 0.17 | 0.06 | 0.06 | 0.270 |
| E. maxima | 22 | 0.85 | 0.79 | 0.88 | 0.975 |
| TMLS | 22 | 1.02 | 0.85 | 0.94 | 0.769 |
| NE percentage | 22 | 90 | 23 | 73 | 0.081 |

Table 2 summarizes the effect of the treatment on gut health scores. The effect of the antibiotic treatment is evident and strong on improving NE scores, E. maxima lesions, and overall coccidiosis lesions (TMLS) in comparison to untreated control on day 21. GAA supplemented birds also had lower amounts of E. maxima and coccidiosis lesions at day 21 and lower NE scores and a lower percentage of NE affected birds at day 22, similar to the antibiotics treatment.

From these results, it can be concluded that GAA supplementation helps the birds to use the energy from feed more effectively to combat NE infection, potentially supporting the energy demanding immune response in a more effective way. In particular, this leads to a reduced mortality from NE, which also increases the overall profitability in real-world conditions. Additionally, body weight gain and feed conversion ratio are improved, while feed intake is not affected. In conclusion, GAA supplementation supports broiler health during an NE challenge and is a probably accost-efficient and animal welfare supporting solution, particularly in commercial “no antibiotics ever” farms.

1. A composition suitable for treatment and/or prophylaxis of a coccidiosis induced condition in poultry, the composition comprising:
guanidinoacetic acid, a salt of guanidinoacetic acid, or a mixture thereof.

2. The composition of claim 1, wherein the coccidiosis is caused by Eimeria spp. and/or Clostridium perfringens.

3. The composition of claim 1, wherein the coccidiosis induced condition is one or more of bacterial enteritis, necrotic enteritis, diarrhea, and lesion.

4. The composition of claim 1, wherein the salt of guanidinoacetic acid is an acid addition salt.

5. The composition of claim 1, wherein the salt of guanidinoacetic acid comprises guanidinoacetic acid hydrogen chloride, guanidinoacetic acid hydrogen sulfate, guanidinoacetic acid hydrogen phosphate, or a mixture of any of these.

6. The composition of claim 1, wherein the composition comprises up to 1,500 ppm of the guanidinoacetic acid, the salt of guanidinoacetic acid, or the mixture thereof.

7. The composition of claim 1, further comprising:
a probiotic.

8. The composition of claim 7, wherein the probiotic comprises spores of Bacillus spp.

9. The composition of claim 7, wherein the probiotic comprises spores of Bacillus amyloliquefaciens and/or Bacillus subtilis.

10. The composition of claim 1, wherein the composition is administered to poultry in any phase, a multitude of phases, or all phases of their lifetime.

11. The composition of claim 1, wherein the composition is administered to poultry starting from the beginning of the starter phase until slaughter or starting from the beginning of the grower phase until slaughter or any other phase of life.

12. The composition of claim 1, wherein the composition is administered to poultry in the grower phase.

13. The composition of claim 1, wherein the composition is a liquid or a solid composition.

14. A diet suitable for the treatment and/or prophylaxis of a coccidiosis induced condition in poultry, wherein the diet comprises the composition of claim 1.
